SEQUENCE LISTING (26 sequences)

SEQ ID NO: 1 - protein, 112 aa, Mus musculus
DIVMTQSTLSLPVSLGDQASISCRSSQSLLYSNGNTYLHWYLQKPGQSPKLLIYKVSNRFSGVPDRFSGSGSGTDFTLKIRRVEAEDLGVYFCSQSRHVPPTFGAGTKLELK

SEQ ID NO: 2 - DNA, 336 bp, Mus musculus
gatattgtgatgacacagtctacactctccctgcctgtcagtcttggagatcaagcctccatctcttgcagatctagtcagagccttctttacagtaatggaaacacctatttacattggtacctccagaagccaggccagtctccaaagctcctgatctacaaagtttccaaccgattttctggggtcccagacaggttcagtggcagtggatcagggacagatttcacactcaagatcagaagagtggaggctgaggatctgggagtttatttctgctctcaaagtagacatgttcctcccacgttcggtgctgggaccaagctggagctgaaa

SEQ ID NO: 3 - protein, 114 aa, Mus musculus
EVKLEQSGPEMVKPGTSVKISCKTSGYAFTDFYMNWVKQSHGNSLEWIGDINPNNGDPTYNQNFKGKATLTVDKSSTTAYIDLRSLTSEDSAVYYCGRGDGAYWGQGTLVTVSA

SEQ ID NO: 4 - DNA, 342 bp, Mus musculus
gaggtaaagctggagcagtcaggacctgagatggtgaagcctgggacctcagtgaagatatcctgtaagacttctggatacgcgttcactgacttttacatgaactgggtgaagcagagccatggaaacagccttgagtggattggagatattaatcctaacaatggtgatcctacctacaaccagaacttcaagggcaaggccacattgactgtagacaagtcctccaccacagcctacatagacctccgcagcctgacatctgaggactctgccgtctattactgtggaagaggggacggggcttactggggccaagggactctggtcactgtctctgca

SEQ ID NO: 5 - DNA, 683 bp, Mus musculus
  misc_feature (671)..(671): n is a, c, g, or t
gaattcgcccttggaagatctcttgtccactttggtgctgcttgccgggtgggccacattgcaggtgatggtctggctgggccaggtgttcgaggttacagtcactgagctgctgagggtgtagaggccagactgcaggagagctgggaaggtgtgcacaccactggacagggatccagagttccaggtcaaggtcactggctcagggaaataacccttgaccaggcatcctagagtcaccgaggagccagttgtacctccacacacaggggccagtggatagaccgatggggctgttgttttggctgaggagactgtgagagtggtgccttggccccagtagtcaaagtagtccgttccaccccatcttgtacaataatagaccgcagagtcctcagatgtcaggctgctgagctgcatgtaggctgtgctggaggatttgactacagtcagcgtggccttgttcttgaaattctcattgtagttagtaacaccaaagcgaggatcaatatttccaatccactcaaggccttgtccaggcctctgcatcatccagtgcatccagtaactggcgaaggtgtagccagaagccttgcaggacagcttcactgaagtcccaggcttcaccagttctgtcccagactcctgcagcttcacctggaattccgganaagggcgaattc

SEQ ID NO: 6 - protein, 227 aa, Mus musculus
  misc_feature (4)..(4): Xaa can be any naturally occurring amino acid
IRPXPEFQVKLQESGTELVKPGTSVKLSCKASGYTFASYWMHWMMQRPGQGLEWIGNIDPRFGVTNYNENFKNKATLTVVKSSSTAYMQLSSLTSEDSAVYYCTRWGGTDYFDYWGQGTTLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKRSSKGEF

SEQ ID NO: 7 - DNA, 458 bp, Mus musculus
  misc_feature (37)..(37): n is a, c, g, or t
  misc_feature (444)..(446): n is a, c, g, or t
gaattcgcccttcttccggaattcgaggttaagctgnaggagtctgggacagaactggtgaagcctgggacttcagtgaagctgtcctgcaaggcttctggctacaccttcgccagttactggatgcactggatgatgcagaggcctggacaaggccttgagtggattggaaatattgatcctcgctttggtgttactaactacaatgagaatttcaagaacaaggccacgctgactgtagtcaaatcctccagcacagcctacatgcagctcagcagcctgacatctgaggactctgcggtctattattgtacaagatggggtggaacggactactttgactactggggccaaggcaccactctcacagtctcctcagccaaaacaacagccccatcggtctatccactggcccctgtgtgtggaggtacaactggctcctcnnngactctaggatg

SEQ ID NO: 8 - protein, 152 aa, Mus musculus
  misc_feature (13)..(13): Xaa can be any naturally occurring amino acid
  misc_feature (148)..(149): Xaa can be any naturally occurring amino acid
EFALLPEFEVKLXESGTELVKPGTSVKLSCKASGYTFASYWMHWMMQRPGQGLEWIGNIDPRFGVTNYNENFKNKATLTVVKSSSTAYMQLSSLTSEDSAVYYCTRWGGTDYFDYWGQGTTLTVSSAKTTAPSVYPLAPVCGGTTGSXXTLG

SEQ ID NO: 9 - DNA, 380 bp, Mus musculus
  misc_feature (88)..(88): n is a, c, g, or t
  misc_feature (100)..(100): n is a, c, g, or t
  misc_feature (105)..(105): n is a, c, g, or t
  misc_feature (164)..(164): n is a, c, g, or t
  misc_feature (169)..(169): n is a, c, g, or t
  misc_feature (172)..(172): n is a, c, g, or t
  misc_feature (174)..(175): n is a, c, g, or t
  misc_feature (214)..(215): n is a, c, g, or t
  misc_feature (223)..(223): n is a, c, g, or t
  misc_feature (226)..(226): n is a, c, g, or t
  misc_feature (230)..(230): n is a, c, g, or t
  misc_feature (241)..(241): n is a, c, g, or t
  misc_feature (252)..(253): n is a, c, g, or t
  misc_feature (273)..(273): n is a, c, g, or t
  misc_feature (276)..(276): n is a, c, g, or t
  misc_feature (294)..(294): n is a, c, g, or t
  misc_feature (299)..(299): n is a, c, g, or t
  misc_feature (302)..(302): n is a, c, g, or t
  misc_feature (305)..(305): n is a, c, g, or t
  misc_feature (315)..(315): n is a, c, g, or t
  misc_feature (323)..(323): n is a, c, g, or t
  misc_feature (328)..(329): n is a, c, g, or t
  misc_feature (338)..(338): n is a, c, g, or t
  misc_feature (362)..(362): n is a, c, g, or t
  misc_feature (365)..(365): n is a, c, g, or t
  misc_feature (367)..(367): n is a, c, g, or t
  misc_feature (372)..(372): n is a, c, g, or t
  misc_feature (374)..(374): n is a, c, g, or t
gaattccccttgaaaatctcttgtccctttggtgctgcttgccgggtgggccacattgcaagtgatggtctggctgggcctgtgttcnaggttacagtcnctganctgctgagggagtccaggccgcactgcaggagagctggtatggtgtgcactccactgcnatcgntancnnagttccaggtcactgactcaggcaaatggacattgaccnngcatcctnaantcancaacaagctanttgtccctcgnnacaccaggcccagtggatanacngatgacgctgttggcttnctgcntanacnctgaccccantccattgncgccnntaagccccntcccctcttaacaataataaacanctnantccgnanatgtca

SEQ ID NO: 10 - DNA, 683 bp, Mus musculus
gaattcgcccttcttccggaattccaagtaaagctgcaggagtctgggacagaactggtgaagcctgggacttcagtgaagctgtcctgcaaggcttctggctacaccttcgccagttactggatgcactggatgatgcagaggcctggacaaggccttgagtggattggaaatattgatcctcgcttggtgttactaactacaatgagaatttcaagaacaaggccacgctgactgtagtcaaatcctccagcacagcctacatgcagctcagcagcctgacatctgaggactctgcggtctattattgtacaagatggggtggaacggactactttgactactggggccaaggcaccactctcacagtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagagatcttccaagggcgaattc

SEQ ID NO: 11 - protein, 218 aa, Mus musculus
EFALLPEFQVKLQESGTELVKPGTSVKLSCKASGYTFASYWMHWMMQRPGQGLEWIGNIDPRLVLLTTMRISRTRPRLSNPPAQPTCSSAAHLRTLRSIIVQDGVERTTLTTGAKAPLSQSPQPKQQPHRSIHWPLCVEVQLAPRLDAWSRVISLSQPPGTLDPCPVVCTPSQLSCSLASTPSAAQLPRTPGPARPSPAMWPTRQAAPKWTRDLPRAN

SEQ ID NO: 12 - DNA, 139 bp, Mus musculus
gaattcgcccttgggagctcgacattgtgatgacccaatctccatctcccatgtatgaatctctaggagagagagtcactatcacttgcaaggggctgatgctgcaccaactgtatccgcatgcaccaagggcgaattc

SEQ ID NO: 13 - DNA, 380 bp, Mus musculus
gaattcgcccttggtgcatgcggatacagttggtgcagcatcagcccctccagcttggtcccagcaccgaacgtgaagaaactcatcatactatagacaataataaattcccatatcttcatagtccaggctggtgatggtgagagaataatcttgcccagatccactgccactgaaccttgatgggaccccatcgaccaatctgtttgcacgatagatcagggtcttaggagatttccctggtttctgctggaaccaacttaaatagctattaatgtcctgactcgccttgcaagtgatagtgactctctctcctagagattcatacatggaagatggagactgtgtgatcacaatatcgagctcccaagggcgaattc

SEQ ID NO: 14 - protein, 89 aa, Mus musculus
DIVITQSPSSMYESLGERVTITCKASQDINSYLSWFQQKPGKSPKTLIYRANRLVDGVPSRFSGSGSGQDYSLTITSLDYEDMGIYYCL

SEQ ID NO: 15 - DNA, 379 bp, Mus musculus
  misc_feature (360)..(360): n is a, c, g, or t
gaattccccttggtgcatgcagatacagttggtgcagcatcagcccctccagcttggtcccagcaccgaacgtggagaaactcatcatactatagacaataataaattcccatatcttcatagtccaggctggtgatggtgagagaataatcttgcccagatccactgccactgaaccttgatgggaccccgtcgaccaatctgtttgcacgatagatcagggtcttaggagatttccctggtttctgctggaaccaacttaaatagctattaatgtcctgactcgtcttgcaagtgatagtgactctctctcctagagattcatacatggaagatgtagtctgggtcagcacaatatcnagctcccaagggcgaattc

SEQ ID NO: 16 - protein, 89 aa, Mus musculus
DIVLTQTTSSMYESLGERVTITCKTSQDINSYLSWFQQKPGKSPKTLIYRANRLVDGVPSRFSGSGSGQDYSLTITSLDYEDMGIYYCL

SEQ ID NO: 17 - DNA, 139 bp, Mus musculus
gaattcgcccttgggagctcgatattgtgctgacacaaactacatcttccatgtatgaatctctaggagagagagtcactatcacttgcaaggggctgatgctgcaccaactgtatccgcatgcaccaagggcgaattc

SEQ ID NO: 18 - protein, 39 aa, Mus musculus
DIVLTQTTSSMYESLGERVTITCKGLMLHQLYPHAPRAN

SEQ ID NO: 19 - DNA, 139 bp, Mus musculus
gaattcgcccttggtgcatgcggatacagttggtgcagcatcagccccttgcaagtgatagtgactctctctcctagagattcatacatggaagatggagattgggtcatcacaatgtcgagctcccaagggcgaattc

SEQ ID NO: 20 - protein, 45 aa, Mus musculus
IRPWELDIVMTQSPSSMYESLGERVTITCKGLMLHQLYPHAPRAN

SEQ ID NO: 21 - DNA, 898 bp, Mus musculus
  misc_feature (2)..(4): n is a, c, g, or t
  misc_feature (21)..(22): n is a, c, g, or t
  misc_feature (822)..(822): n is a, c, g, or t
  misc_feature (836)..(836): n is a, c, g, or t
gnnnttggtaccgagctcggnnccctagtaacggccgccagtgtgctggaattcgcccttggtgcatgcggatacagttggtgcagcatcagcccctccagcttggtcccagcaccgaacgtgaagaaactcatcatactatagacaataataaattcccatatcttcatagtccaggctggtgatggtgagagaataatcttgcccagatccactgccactgaaccttgatgggaccccatcgaccaatctgtttgcacgatagatcagggtcttaggagatttccctggtttctgctggaaccaacttaaatagctattaatgtcctgactcgccttgcaagtgatagtgactctctctcctagagattcatacgtggaagatgtagtctgggtcagcacaatatcgagctcccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcnggggctccctttanggttccgatttagtgctttacggcacctcgaccccaaaaaacttgattagggtgatggttca

SEQ ID NO: 22 - DNA, 907 bp, Mus musculus
  misc_feature (1)..(6): n is a, c, g, or t
  misc_feature (9)..(9): n is a, c, g, or t
  misc_feature (11)..(11): n is a, c, g, or t
  misc_feature (13)..(13): n is a, c, g, or t
  misc_feature (21)..(21): n is a, c, g, or t
  misc_feature (835)..(835): n is a, c, g, or t
  misc_feature (906)..(906): n is a, c, g, or t
nnnnnnggnancnagctcggntccctagtaacggccgccagtgtgctggaattcgcccttggtgcatgcggatacagttggtgcagcatcagcccctccagcttggtcccagcaccgaacgtgaagaaactcatcatactatagacaataataaattcccatatcttcatagtccaggctggtgatggtgagagaataatcttgcccagatccactgccactgaaccttgatggaccccatcgaccaatctgtttgcacgatagatcagggtcttaggagatttccctggtttctgctggaaccaacttaaatagctattaatgtcctgactcgccttgcaagtgatagtgactctctctcctagagattcatacatggaagatggagactgggtcagcacaatatcgagctcccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttanggttccgatttagtgctttacggcacctcgaccccaaaaaacttgattagggtgatggttcacgtagtggnc

SEQ ID NO: 23 - DNA, 907 bp, Mus musculus
  misc_feature (1)..(5): n is a, c, g, or t
  misc_feature (8)..(8): n is a, c, g, or t
  misc_feature (20)..(21): n is a, c, g, or t
  misc_feature (107)..(107): n is a, c, g, or t
  misc_feature (118)..(118): n is a, c, g, or t
  misc_feature (131)..(131): n is a, c, g, or t
  misc_feature (145)..(149): n is a, c, g, or t
  misc_feature (159)..(159): n is a, c, g, or t
  misc_feature (166)..(166): n is a, c, g, or t
  misc_feature (174)..(176): n is a, c, g, or t
  misc_feature (180)..(180): n is a, c, g, or t
  misc_feature (189)..(189): n is a, c, g, or t
  misc_feature (194)..(194): n is a, c, g, or t
  misc_feature (212)..(213): n is a, c, g, or t
  misc_feature (217)..(217): n is a, c, g, or t
  misc_feature (229)..(229): n is a, c, g, or t
  misc_feature (241)..(241): n is a, c, g, or t
  misc_feature (267)..(267): n is a, c, g, or t
  misc_feature (309)..(309): n is a, c, g, or t
  misc_feature (311)..(311): n is a, c, g, or t
  misc_feature (363)..(364): n is a, c, g, or t
  misc_feature (367)..(367): n is a, c, g, or t
  misc_feature (377)..(378): n is a, c, g, or t
  misc_feature (380)..(380): n is a, c, g, or t
  misc_feature (382)..(382): n is a, c, g, or t
  misc_feature (408)..(408): n is a, c, g, or t
  misc_feature (426)..(427): n is a, c, g, or t
  misc_feature (437)..(437): n is a, c, g, or t
  misc_feature (448)..(448): n is a, c, g, or t
  misc_feature (496)..(497): n is a, c, g, or t
  misc_feature (539)..(540): n is a, c, g, or t
  misc_feature (560)..(560): n is a, c, g, or t
  misc_feature (591)..(591): n is a, c, g, or t
  misc_feature (604)..(604): n is a, c, g, or t
  misc_feature (624)..(625): n is a, c, g, or t
  misc_feature (630)..(631): n is a, c, g, or t
  misc_feature (634)..(635): n is a, c, g, or t
  misc_feature (641)..(645): n is a, c, g, or t
  misc_feature (647)..(647): n is a, c, g, or t
  misc_feature (649)..(650): n is a, c, g, or t
  misc_feature (652)..(652): n is a, c, g, or t
  misc_feature (654)..(655): n is a, c, g, or t
  misc_feature (663)..(663): n is a, c, g, or t
  misc_feature (665)..(665): n is a, c, g, or t
  misc_feature (670)..(670): n is a, c, g, or t
  misc_feature (672)..(674): n is a, c, g, or t
  misc_feature (676)..(677): n is a, c, g, or t
  misc_feature (683)..(683): n is a, c, g, or t
  misc_feature (688)..(690): n is a, c, g, or t
  misc_feature (692)..(692): n is a, c, g, or t
  misc_feature (708)..(708): n is a, c, g, or t
  misc_feature (710)..(710): n is a, c, g, or t
  misc_feature (713)..(713): n is a, c, g, or t
  misc_feature (715)..(715): n is a, c, g, or t
  misc_feature (718)..(719): n is a, c, g, or t
  misc_feature (725)..(725): n is a, c, g, or t
  misc_feature (739)..(739): n is a, c, g, or t
  misc_feature (741)..(747): n is a, c, g, or t
  misc_feature (749)..(749): n is a, c, g, or t
  misc_feature (757)..(757): n is a, c, g, or t
  misc_feature (764)..(764): n is a, c, g, or t
  misc_feature (772)..(772): n is a, c, g, or t
  misc_feature (777)..(777): n is a, c, g, or t
  misc_feature (785)..(786): n is a, c, g, or t
  misc_feature (788)..(788): n is a, c, g, or t
  misc_feature (791)..(791): n is a, c, g, or t
  misc_feature (796)..(797): n is a, c, g, or t
  misc_feature (799)..(799): n is a, c, g, or t
  misc_feature (812)..(812): n is a, c, g, or t
  misc_feature (821)..(821): n is a, c, g, or t
  misc_feature (824)..(824): n is a, c, g, or t
  misc_feature (832)..(833): n is a, c, g, or t
  misc_feature (839)..(839): n is a, c, g, or t
  misc_feature (843)..(843): n is a, c, g, or t
  misc_feature (845)..(847): n is a, c, g, or t
  misc_feature (852)..(852): n is a, c, g, or t
  misc_feature (856)..(856): n is a, c, g, or t
  misc_feature (858)..(858): n is a, c, g, or t
  misc_feature (861)..(862): n is a, c, g, or t
  misc_feature (868)..(868): n is a, c, g, or t
  misc_feature (876)..(876): n is a, c, g, or t
  misc_feature (880)..(880): n is a, c, g, or t
  misc_feature (884)..(885): n is a, c, g, or t
  misc_feature (888)..(888): n is a, c, g, or t
  misc_feature (890)..(893): n is a, c, g, or t
  misc_feature (896)..(898): n is a, c, g, or t
  misc_feature (904)..(905): n is a, c, g, or t
nnnnnggnaccgagctcggnnccctagtaacggccgccagtgtgctggaattcgcccttggtgcatgcggatacagttggtgcagcatcagccccttgcacttgatnccgactctctntcctaaaaattcntacatggtagatgnnnnntgaattccanaatgtcnagctccannnggcnaattctganataanaatcatgctggcatccgnncgancatgcatctgangggaccaattcnacctatctggattcacaatacatctnggtggcaggagatttacctggtctcggctggaacaaccttgncnttacctaactgacccgacttgccttgcatccgattttgaccagctggcgtanngantaatacgccnnanangatcaccgttccaaacagttgcgcancctgaatgggcaatgganncgccctgtancggcacagtcngcgcggcgggcgtgctggttacgcgcagcgtgaccgctatacttgccnncgccataactccctgtcctttcttttacttcccttcctttgnnaaaacttcgccggctttccncttaaagctcttgctcgggggccccctttanggttctgatttantgctttacaggccctcaccnntcgcnnatnngattannnnnangnntnanntggtgaangnacgcncnnngnnacggcncattnnncnttgacgttgtgagtgntnacncncanntagtgnactcttgttgccancnnnnnnncncctctccntatcttnctctattnttttnatttatanngnatnctgcnnanctcgtcctactgnataacaaanganctgattanngatacnattnannncaatnctgncncanntcccgncgcaacgnctgntagnngangnnnnacnnntagaanncc

SEQ ID NO: 24 - DNA, 924 bp, Mus musculus
  misc_feature (1)..(3): n is a, c, g, or t
  misc_feature (20)..(21): n is a, c, g, or t
  misc_feature (922)..(924): n is a, c, g, or t
nnnttggtaccgagctcggnnccctagtaacggccgccagtgtgctggaattcgcccttggtgcatgcggatacagttggtgcagcatcagcccctccagcttggtcccagcaccgaacgtgaagaaactcatcatactatagacaataataaattcccatatcttcatagtccaggctggtgatggtgagagaataatcttgcccagatccactgccactgaaccttgatgggaccccatcgaccaatctgtttgcacgatagatcagggtcttaggagatttccctggtttctgctggaaccaacttaaatagctattaatgtcctgactcgccttgcaagtgatagtgactctctctcctagagattcatacatggaagatggagtctgggtcatcacaatatcgagctcccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaacttgattagggtgatggttcacgtagtgggccatcgccctgatagannn

SEQ ID NO: 25 - DNA, 909 bp, Mus musculus
  misc_feature (1)..(5): n is a, c, g, or t
  misc_feature (14)..(14): n is a, c, g, or t
  misc_feature (22)..(23): n is a, c, g, or t
  misc_feature (846)..(846): n is a, c, g, or t
  misc_feature (895)..(895): n is a, c, g, or t
  misc_feature (905)..(905): n is a, c, g, or t
  misc_feature (909)..(909): n is a, c, g, or t
nnnnnttggtaccnagctcggnnccctagtaacggccgccagtgtgctggaattcgcccttggtgcatgcggatacagttggtgcagcatcagcccgtttcagctccagcttggtcccagcaccgaacgtgagcggataattccaatgctgcagacagaaataatctgccaggtcttcagattgcacactgccaatggtgagagtgaaatctgtcccagatccactgcctgtgaagcgatcagggactccagtgtgccggttggatgtcaagtaaatcagtgctttaggagactgtcctggtttctgtcgataccaggctacagcagtacgaacattctgactggccttgcaggtgatgctgaccctgtctcctactgatgtggacatgaattttggagtttgggtcatcacaatgtcgagctcccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttanggttccgatttagtgctttacggcacctcgaccccaaaaaacttgattngggtgatggntcan

SEQ ID NO: 26 - DNA, 910 bp, Mus musculus
  misc_feature (1)..(5): n is a, c, g, or t
  misc_feature (21)..(21): n is a, c, g, or t
  misc_feature (800)..(802): n is a, c, g, or t
nnnnntggtaccgagctcggntccctagtaacggccgccagtgtgctggaattcgcccttggtgcatgcggatacagttggtgcagcatcagcccctccagcttggtcccagcaccgaacgtgaagaaactcatcatactatagacaataataaattcccatatcttcatagtccaggctggtgatggtgagagaataatcttgcccagatccactgccactgaaccttgatgggaccccatcgaccaatctgtttgcacgatagatcagggtcttaggagatttccctggtttctgctggaaccaacttaaatagctattaatgtcctgactcgccttgcaagtgatagtgactctctctcctagagattcatacatggaagatggagattgtgtcatcacaatatcgagctcccaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggcnnncccgtcaagctctaaatcgggggctccctttagggttccgatttagtgctttacggcacctcgaccccaaaaaacttgattagggtgatggttcacgtagtgggccat

(19) United States
(12) Patent Application Publication
(10) Pub. No.: US 2022/0332805 A1
(43) Pub. Date: Oct. 20, 2022

(54) Treatment of Skin Blistering Diseases Using Antibodies

(71) Applicant: University of Cincinnati, Cincinnati, OH (US)

(72) Inventors: Fred Finkelman, Netanya (IL); Unni Samavedam, Cincinnati, OH (US); Crystal Potter, Fairfield, OH (US); Ralf Ludwig, Gros Gronau (DE); Anika Kasprick, Lubeck (DE); Katja Bieber, Lubeck (DE); Elizabeth Angerman, Cincinnati, OH (US)

(73) Assignee: University of Cincinnati, Cincinnati, OH (US)

(21) Appl. No.: 17/641,297

(22) PCT Filed: Sep. 11, 2020
(86) PCT No.: PCT/US20/50538

§ 371 Date: Mar. 8, 2022

Related U.S. Application Data
(60) Provisional application No. 62/898,830, filed on Sep. 11, 2019.

(51) Int. Cl. C07K 16/18; A61P 17/00; A61K 47/00

(52) U.S. Cl. CPC C07K 16/18; A61P 17/00; A61K 47/00

(57) ABSTRACT

Methods and compositions for treating a skin blistering disease are disclosed. In one embodiment, a method of treating a subject suffering from a skin blistering disease is disclosed. The method involves identifying an epithelial cell adhesion molecule associated with the skin blistering disease (EpCAM); then identifying a non-pathogenic monoclonal antibody (mAb) that targets the EpCAM; and then administering to the subject a therapeutically effective amount of the mAb.

### CROSS REFERENCE TO RELATED APPLICATIONS

[1] This application claims priority to U.S. Provisional Application Ser. No. 62/898,830, filed Sep. 11, 2019, and PCT application PCT/US20/50538, filed Sep. 11, 2020, which applications are hereby incorporated by reference in their entirety.

### STATEMENT REGARDING FEDERALLY FUNDED RESEARCH OR DEVELOPMENT

This invention was made with government support under R01 AI130103 awarded by the National Institutes of Health. The U.S. Government has certain rights in the invention.

### TECHNICAL FIELD

The present invention relates to a method of treating skin blistering diseases.

### BACKGROUND OF THE INVENTION

Skin blistering diseases are associated with severe morbidity, as they result in denudation of skin and mucous membranes. Symptoms include severe itching, pain, weeping and oozing of the skin, malnutrition, and infection. Untreated, these disorders can lead to significant mortality risk. Treatment of these disorders is largely non-specific and involves long-term systemic corticosteroids and immunosuppressive agents. However, long-term treatment with these drugs often only partially controls the symptoms and may cause many dose-related adverse effects. Currently, there is an unmet medical need for therapies that can treat these serious diseases in a safe and effective manner SUMMARY OF THE INVENTION

The present invention provides, among other things, methods and compositions for treating a skin blistering disease. In one embodiment, a method of treating a subject suffering from a skin blistering disease is disclosed. The method involves identifying an epithelial cell adhesion molecule associated with the skin blistering disease (EpCAM); then identifying a non-pathogenic monoclonal antibody (mAb) that targets the EpCAM; and then administering to the subject a therapeutically effective amount of the mAb. In another embodiment, the skin disease is epidermolysis bullosa acquistita (EBA). In one embodiment, the epithelial cell adhesion molecule is collagen type VII. In another embodiment, the mAb is an IgG2c anti-collagen type VII mAb. In one embodiment, the mAb is an antibody that binds to an epithelial cell adhesion molecule (EpCAM) comprising a heavy chain having the amino acid sequence SEQ ID NO:3, and a light chain having the amino acid sequence SEQ ID NO:1.

In one embodiment of the present invention, the skin disease is bullous pemphigoid (BP). In another embodiment, the epithelial cell adhesion molecule is collagen type XVII. In one embodiment, the mAb further includes a pharmaceutically acceptable carrier, excipient, flow agent, processing aid, diluent or a combination thereof.

In another embodiment, a method of treating a subject with a skin blistering disease is disclosed. The method involves administering to the subject a therapeutically effective amount of a pharmaceutical composition comprising a non-pathogenic monoclonal antibody (mAb) and a pharmaceutically acceptable carrier excipient, flow agent, processing aid, diluent or a combination thereof to the subject. In one embodiment, the mAb is an IgG2c anti-collagen type VII mAb. In another embodiment, the mAb is an antibody that binds to an epithelial cell adhesion molecule (EpCAM) comprising a heavy chain having the amino acid sequence SEQ ID NO:3, and a light chain having the amino acid sequence SEQ ID NO:1.

In one embodiment, a composition for the treatment of a skin blistering disease is disclosed. The composition comprises a non-pathogenic monoclonal antibody (mAb), a pharmaceutically acceptable carrier, excipient, flow agent, processing aid, diluent or a combination thereof. In another embodiment, the mAb is an IgG2c anti-collagen type VII mAb. In one embodiment, the mAb is an antibody that binds to an epithelial cell adhesion molecule (EpCAM) comprising a heavy chain having the amino acid sequence SEQ ID NO:3, and a light chain having the amino acid sequence SEQ ID NO:1. In one embodiment, the composition is in a form suitable for oral, intravenous, intraaorterial, intramuscular, subcutaneous, parenteral, transmucosal, transdermal, ocular, or topical administration. In another embodiment, the composition is a topical application in the form of a cream, an ointment, a suspension, an emulsion, a gel or a combination thereof.

### BRIEF DESCRIPTION OF THE DRAWINGS

The foregoing summary, as well as the following detailed description of preferred embodiments of the application, will be better understood when read in conjunction with the appended drawings.

FIG. 1 is a pair of graphs showing how IgG1 deficiency increases the risk of active mEBA.

FIG. 2 is a series of graphs showing that IgG1 suppresses the development of active mEBA.

FIG. 3 is a graph showing that IFN-7 is not required for development of active mEBA.

FIG. 4 is a graph showing suppression of induction of passive mEBA with a combination of two anti-mouse Col7 mAbs.

FIG. 5 is a graph showing that mAb 16A1C8, but not mAb 9D8H4, suppresses passive model of mEBA.

FIG. 6 is a graph showing dose-related suppression of passive EBA by 16A1C8.

FIG. 7 is a graph showing that suppression of EBA Induction by mAb 16A1C8 is FcγRIIB-independent.

FIG. 8 is a pair of graphs showing that treatment with mAb 16A1C8 decreased disease progression and promoted lesion healing when administered to mice that had established mEBA.

FIG. 9 identifies the light variable and heavy variable for amino acids and V region DNA sequences for mAb 16A1C8. Specifically, SEQ ID NO:1, SEQ ID NO 2, SEQ ID NO:3 and SEQ ID NO4.

FIGS. 10-28 identify the amino acids and DNA sequences for variations of mAb 9D8H4. Specifically, SEQ ID NO:5, SEQ ID NO 6, SEQ ID NO:7, SEQ ID NO:8, SEQ ID NO 9, SEQ ID NO:10, SEQ ID NO:11, SEQ ID NO 12, SEQ ID NO:13, SEQ ID NO:14, SEQ ID NO 15, SEQ ID NO:16, SEQ ID NO:17, SEQ ID NO 18, SEQ ID NO:19, SEQ ID NO:20, SEQ ID NO 21, SEQ ID NO:22, SEQ ID NO:23, SEQ ID NO 24, SEQ ID NO:25 and SEQ ID NO:26.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

A “dosing regimen” (or “therapeutic regimen”), as that term is used herein, is a set of unit doses (typically more than one) that are administered individually to a subject, typically separated by periods of time. In some embodiments, a given therapeutic agent (e.g., a mAb) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

“EpCAM” means an “epithelial cell adhesion molecule” that has the property of adhesion to epithelial cells. It is widely expressed on epithelial origin cells and epithelial derived tumor cells.

As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

As used herein, the term “prevent” or “prevention”, when used in connection with the occurrence of a disease, disorder, and/or condition, refers to reducing the risk of developing the disease, disorder and/or condition. See the definition of “risk.”

The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

As will be understood from context, a “risk” of a disease, disorder, and/or condition comprises a likelihood that a particular individual will develop a disease, disorder, and/or condition (e.g., epidermolysis bullosa acquistita). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., epidermolysis bullosa). In some embodiments a reference sample or group of reference samples are from individuals comparable to a particular individual. In some embodiments, relative risk is 0, 1, 2, 3, 4, 5, 6, 7, 8, 9, 10, or more.

As used herein, the term “sign” refers to a departure from normal body function that indicates the presence of a disease or abnormality that is noticed by a person other than the patient (as opposed to a symptom, see below).

As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith. In the context of a formulation a stable formulation is one in which the therapeutic agent therein essentially retains its physical and/or chemical integrity and biological activity upon storage and during processes (such as freeze/thaw, mechanical mixing and lyophilization).

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

An individual who is “suffering from” a disease, disorder, and/or condition has been diagnosed with or displays one or more signs and/or symptoms of the disease, disorder, and/or condition.

An individual who is “susceptible to” a disease, disorder, and/or condition has not been diagnosed with the disease, disorder, and/or condition. In some embodiments, an individual who is susceptible to a disease, disorder, and/or condition may not exhibit signs and/or symptoms of the disease, disorder, and/or condition. In some embodiments, an individual who is susceptible to a disease, disorder, condition, or event (for example, epidermolysis bullosa) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

As used herein, the term “symptom” refers to a departure from normal body function that indicates the presence of a disease or abnormality that is noticed by the subject or patient.

As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs or symptoms of a disease and/or exhibits only early signs and/or symptoms of the disease for the purpose of decreasing the risk of developing pathology associated with the disease.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention concerns a method of treating blistering skin diseases. Non-limiting examples of such skin blistering diseases include epidermolysis bullosa acquistita (EBA), pemphigoid disease (i.e., bullous pemphigoid, mucous membrane pemphigoid, or pemphigoid gestationis), IgA-mediated bullous dermatoses (i.e., dermatitis herpetiformis or linear IgA bullous dermatosis), and pemphigus disease (i.e., IgA pemphigus, pemphigus vulgaris, or pemphigus foliaceus). In one embodiment, the present invention concerns a method of treating the blistering skin disease, epidermolysis bullosa acquisita (EBA). EBA is a chronic inflammatory, mucocutaneous blistering skin diseases caused by autoantibodies against the structural skin protein, collagen type VII (Col7). EBA induction requires autoantibody binding as well as complement- and Fcγ receptor (FcγR)-dependent inflammation. Another embodiment concerns the use of a particular immunoglobulin G (IgG) isotype of an anti-Col7 antibody for inhibiting EBA. In a specific embodiment, IgG2c anti-Col7 mAbs 16A1C8 (which is an antibody that binds to an epithelial cell adhesion molecule (EpCAM) comprising a heavy chain having the amino acid sequence SEQ ID NO:3, and a light chain having the amino acid sequence SEQ ID NO:1.) is used to inhibit EBA.

The present invention concerns autoimmune blistering skin diseases that are caused by pro-inflammatory antibodies to a molecule expressed by epithelial cells that promote adhesion of epithelial cells to each other. Although the various autoimmune blistering skin diseases differ in the specific epithelial cell molecule that is targeted by autoantibodies and, as a result, in the precise anatomical location of blister formation, these diseases share the requirement for production of antibodies that both bind to an epithelial cell adhesion molecule and induce inflammation by activating the complement cascade and myeloid cell Fc receptors. The present invention has surprisingly discovered that a mAb to an epithelial adhesion molecule associated with a particular skin blistering disease will block the induction of that blistering skin disease that is normally induced by autoantibodies to that adhesion molecule. For example, we discovered that a non-pathogenic mAb to Col7 blocks the induction of EBA. It is possible that a similar approach could block the induction of the most common autoimmune blistering skin disease, bullous pemphigoid, which is caused by autoantibodies to collagen type XVII (Col17), Murine bullous pemphigoid (mBP) can be induced by passive immunization, using a protocol in which BALB/c or C57BL/6 mice are injected subcutaneously every other day for 12 days with 15 mg of the IgG fraction of an already described rabbit anti-Col17 antiserum (made against a fusion protein that combines amino acids 497 to 573 of mCol17 with glutathione-S-transferase (GST)). Consequently, we could evaluate the ability of one or more non-pathogenic mAbs to the same or different parts of Col17 to block rabbit anti-Col17 induction of mBP or to suppress mBP that had already been induced by injection of the rabbit antibody.

### Skin Blistering Diseases

While not intended to be limiting, some of the skin blistering diseases that may be treated or prevented with the present invention are described below.

### Epidermolysis Bullosa Acquisita (EBA)

Epidermolysis bullosa acquisita (EBA) is a rare type of epidermolysis bullosa that isn't inherited. It is a chronic autoimmune subepidermal blistering disease of the skin and mucus membranes that can occur in people of all ages. Initial manifestations are highly variable, sometimes resembling those of bullous pemphigoid. Bullous lesions are most often in areas subject to minor trauma, such as the extensor aspects of the elbows and the dorsal aspects of the hands and feet. Healing usually causes scars, milia (superficial epidermal inclusion cysts), and hyperpigmentation. However, a subset of EBA patients has a generalized inflammatory skin blister phenotype. Immunologically, EBA is characterized by the presence of immunoglobulin G (IgG) autoantibodies (in most patients) targeting the noncollagenous (NCl) domain of type VII collagen, the major component of anchoring fibrils that connect the basement membrane to dermal structures. The loss of anchoring fibrils leads to the formation of blisters just under the epidermis within an area known as the lamina densa.

Some patients with EBA have been reported to have other health problems, most often Crohn's disease, systemic lupus erythematosus, amyloidosis, multiple myeloma and rarely carcinoma of the lung and lymphoma. Other patients only have a skin problem. The reason why autoantibodies are produced is unknown.

### Pemphigus

Pemphigus is a group of rare autoimmune skin disorders characterized by the development of blisters in the outer layer of the skin (epidermis) and mucous membranes (thin moist layers that line the body's internal surfaces). The location and type of blisters vary according to the type of pemphigus. If left untreated, pemphigus may be a serious illness. Blisters in the outer layer of the skin are common to all types of pemphigus. Blisters develop due to the destruction of the “cement” that holds cells together (epidermal acantholysis), resulting in the separation of cells from one another. Soft (flaccid) blisters generally occur on the neck, scalp, mucous membranes, and/or underarm (axillary) and groin areas (inguinal). Most patients with pemphigus have deposits of IgG around the blistered areas in the epidermal cells called keratinocytes. Anti-epidermal antibodies directed against skin cells are typically present in the fluid of the blisters. The diagnosis of pemphigus requires microscopic examination of cells in the blisters, as well as detection of the IgG antibodies that characterize this disease.

Bullous pemphigoid is a rare, chronic condition in which fluid-filled blisters (bullae) erupt on the surface of the skin, usually the arms, legs or trunk of the affected individual. The cause of bullous pemphigoid is related to the deposition of an IgG antibody directed towards the bullous pemphigoid antigen (collagen type XVII) within the skin.

### Carrier

In one embodiment, the invention provides a composition for the treatment of skin blistering disease, comprising a non-pathogenic monoclonal antibody, a pharmaceutically acceptable carrier, excipient, flow agent, processing aid, diluent or a combination thereof, wherein the composition is in a form suitable for oral, intravenous, intraarterial, intramuscular, subcutaneous, parenteral, transmucosal, transdermal, ocular, or topical administration. In one embodiment, the non-pathogenic monoclonal antibody is present at a therapeutically effective amount that is adjusted specifically to the disease or disease severity sought to be treated.

As used herein, composition means therapeutically effective amounts of the agent together with suitable diluents, preservatives, solubilizers, emulsifiers, adjuvant and/or carriers. A “therapeutically effective amount” as used herein refers to that amount that provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), antioxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors or permeation enhancers for various modes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein pharmaceutically acceptable carriers are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, foams, aerosols, sprays, suspensions, gels, creams, ointments and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present such as, for example, antimicrobials, antioxidants, chelating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions of the invention incorporate particulate forms, protective coatings, protease inhibitors or permeation enhancers for various routes of administration, including parenteral, pulmonary, nasal and oral.

### Combination Therapies

In some embodiments, the non-pathogenic monoclonal antibodies (mAbs) of the present invention will be used as a part of a combination therapy. In some embodiments, mAbs may be administered prior to, concurrently with, or subsequent to one or more additional therapies. It is contemplated that any known therapy or therapeutic for the treatment of epidermolysis bullosa acquisita may be used with one or more non-pathogenic monoclonal antibodies as disclosed herein. Exemplary therapies that may be used with one or more non-pathogenic monoclonal antibodies include, but are not limited to, medications that can help control pain and itching, medications that address complications such as sepsis (e.g., antibiotics), medications that reduce inflammation (e.g., corticosteroid), protein replacement therapy, cell-based therapies, and/or combinations thereof, among others.

### Kits

In some embodiments, the present invention further provides kits or other articles of manufacture that contains non-pathogenic monoclonal antibodies or a formulation containing the same and provides instructions for its reconstitution (if lyophilized) and/or use. Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may hold one or more formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, a container may contain a single dose of a stable formulation containing non-pathogenic monoclonal antibodies. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

### Experiment Discussion

Two sets of experiments with a mouse model were conducted (see the Examples). The first set of experiments (Examples 1-3) used an active disease model in which murine EBA (mEBA) was induced by injecting genetically susceptible mice with a collagen type VII (Col7) fragment plus adjuvant. These studies demonstrated that mice that lack the ability to produce the IgG1 immunoglobulin isotype develop more severe disease than genetically identical mice that can produce IgG1. The studies also demonstrated that the inability to produce IgG1 is not associated, in this model, with increased production of other IgG isotypes (IgG2a (called IgG2c in C57BL/6 mice), IgG2b, IgG3) of anti-collagen type VII (Col7) antibody. In addition, these studies showed that a neutralizing monoclonal antibody to the cytokine IFN-γ has little or no effect on EBA in this model, even in IgG1-deficient mice, although IFN-γ is usually important for an IgG2c antibody response. Taken together, the examples show that the isotype of antibody produced against Col7 affects the disease phenotype, with IgG1 antibodies playing a negative regulatory role.

The second set of experiments (Examples 4-7) uses a passive disease model in which mEBA was induced by injecting BALB/c or C57BL/6 mice with the IgG fraction of a rabbit anti-Col7 (RaCol7) antiserum (rabbits produce only one IgG isotype). After discovering that neither of two mouse IgG2c anti-col7 mAbs (16A1C8 and 9D8H4) induced mEBA when injected into mice, alone or together, we evaluated whether they would affect RaCol7-induced EBA. Whether injected prior to or along with RaCol7, the combination of mouse anti-Col7 mAbs almost completely prevented disease. 16A1C8 was much more effective at suppressing passive mEBA than 9D8H4, which had little effect, and 100 μg of 16A1C8 was considerably more effective at suppressing disease than a combination of 50 μg each of the two mAbs, which appear to bind to partially overlapping Col7 epitopes. A dose response study demonstrated that 100 μg doses of 16A1C8 considerably suppressed RaCol7-induced EBA, while 200 μg doses of 16A1C8 caused almost complete suppression. Mice that lack the inhibitory IgGFc receptor, FcγRIB, developed more severe mEBA than FcγRIIB-sufficient mice in response to RaCol7; however, suppression of mEBA by mAb 16A1C8 in this model is FcγRIIB-independent.

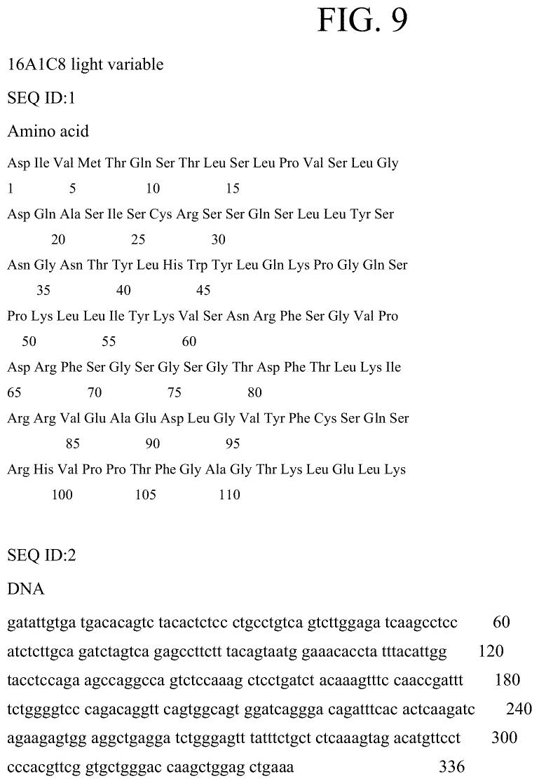

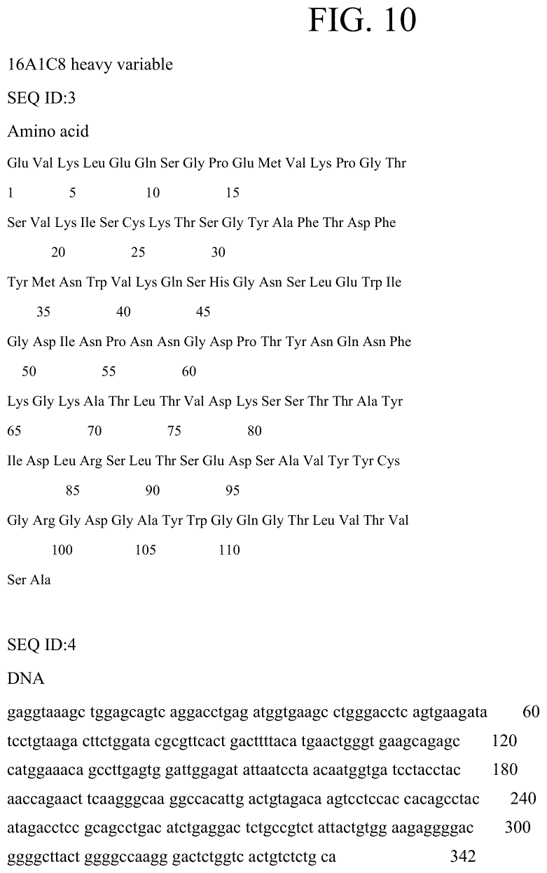

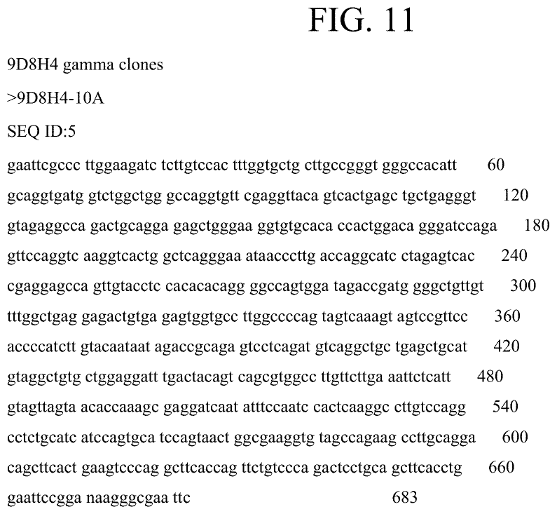

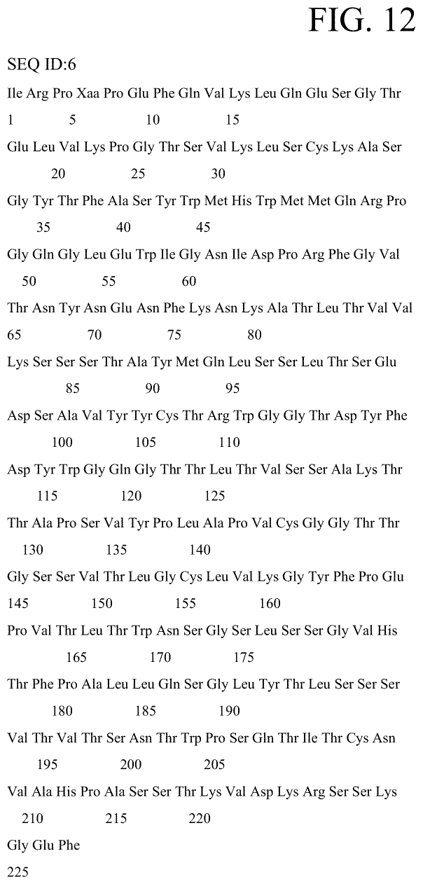

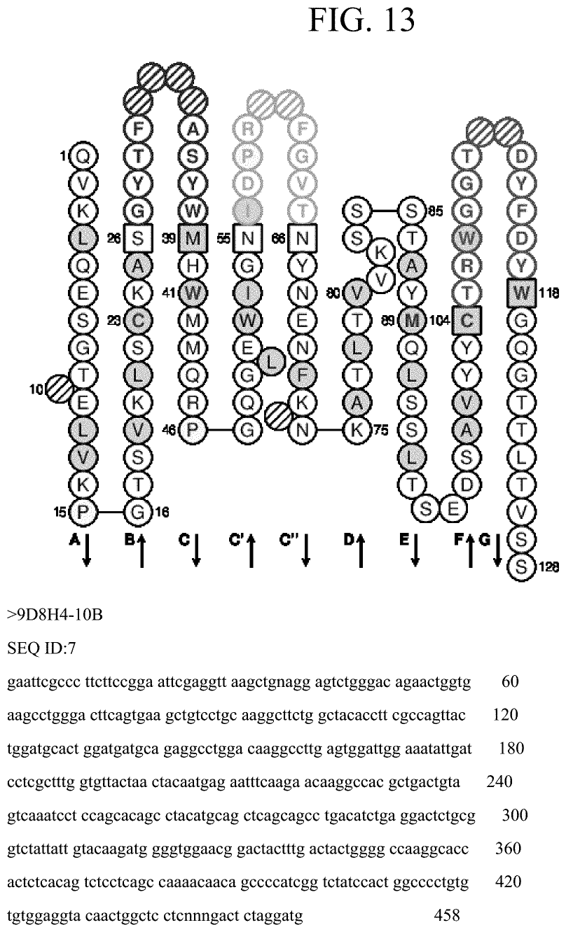

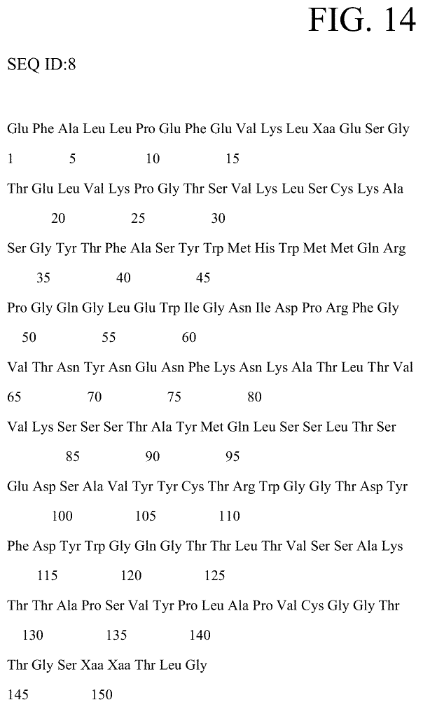

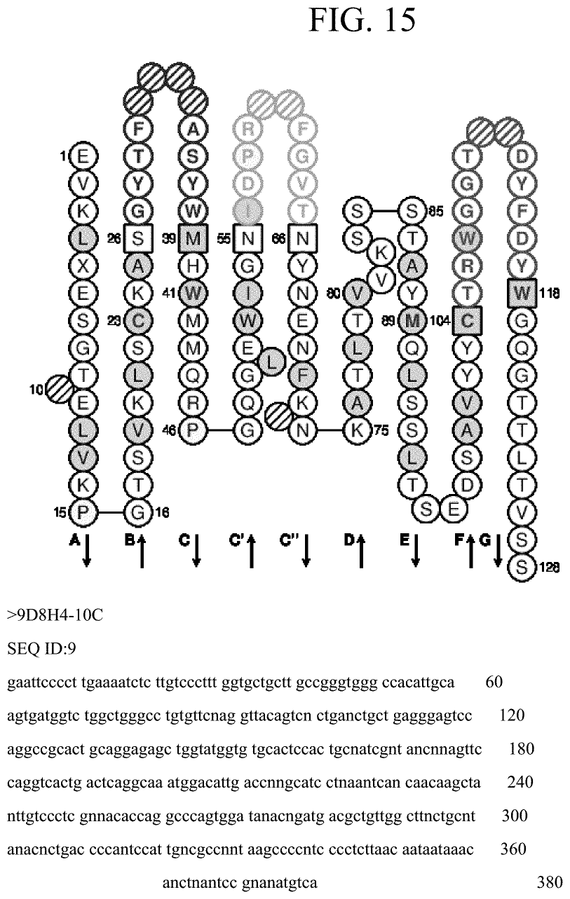

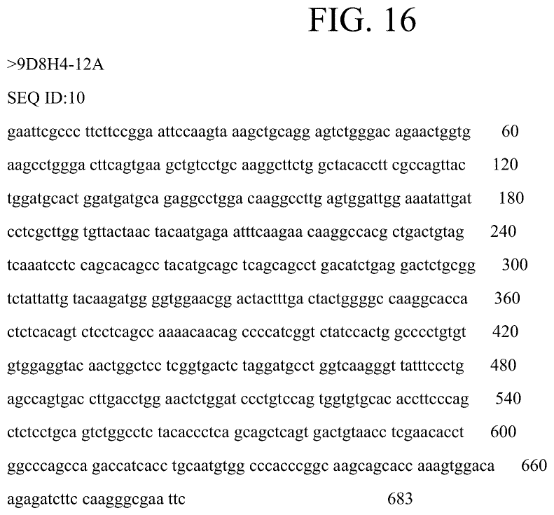

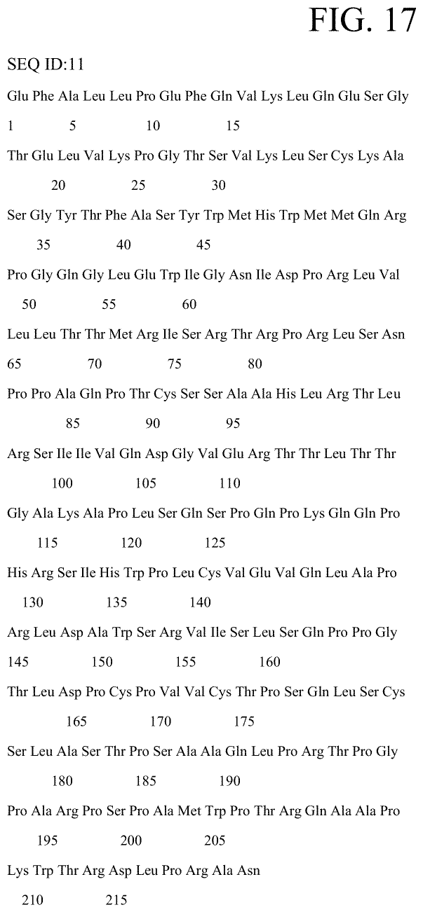

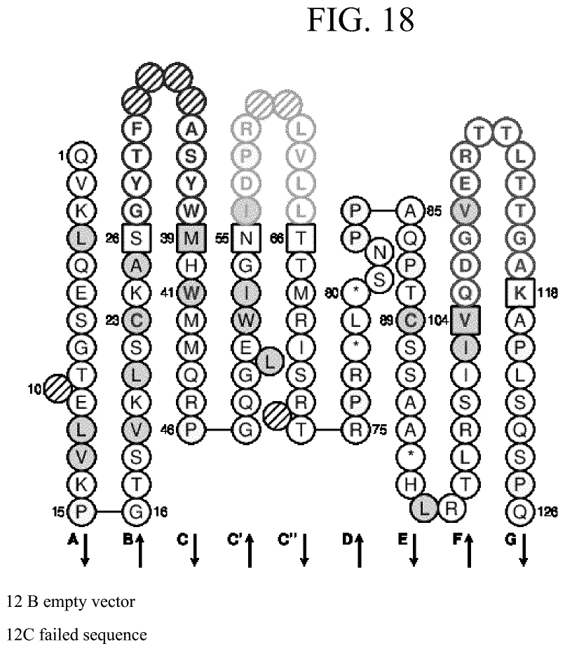

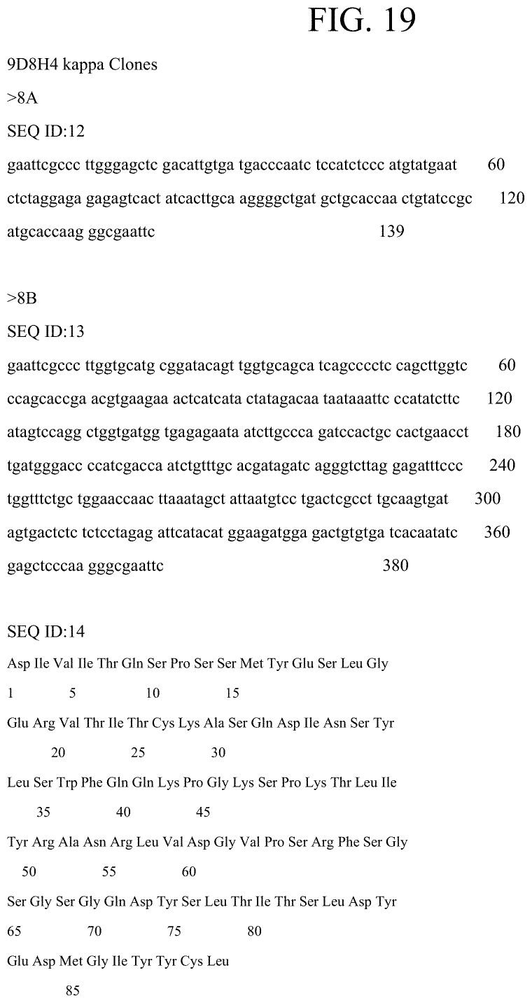

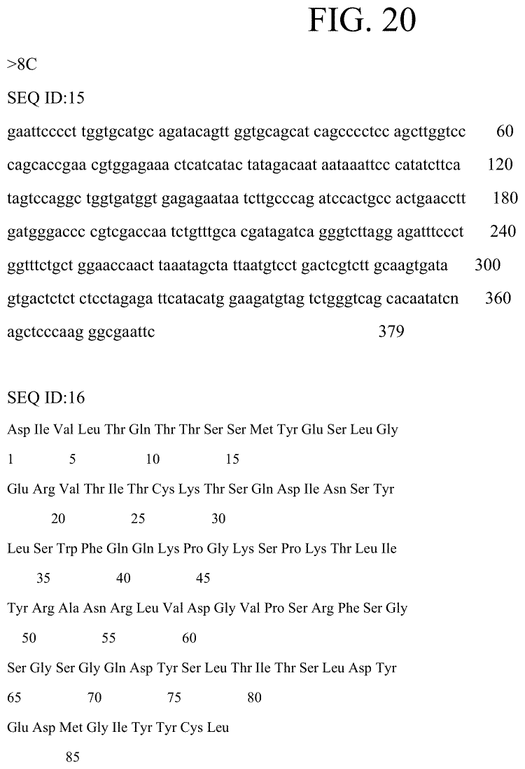

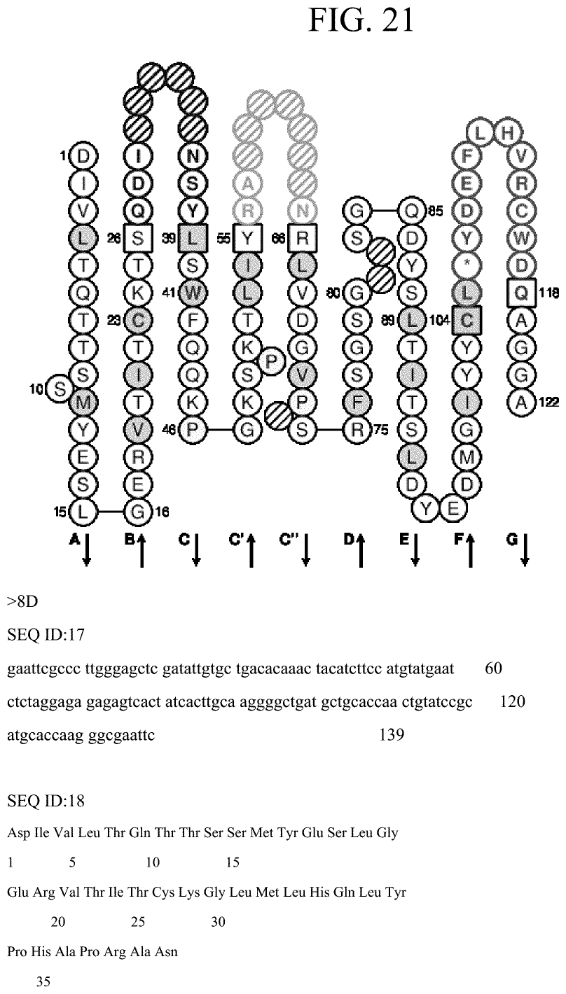

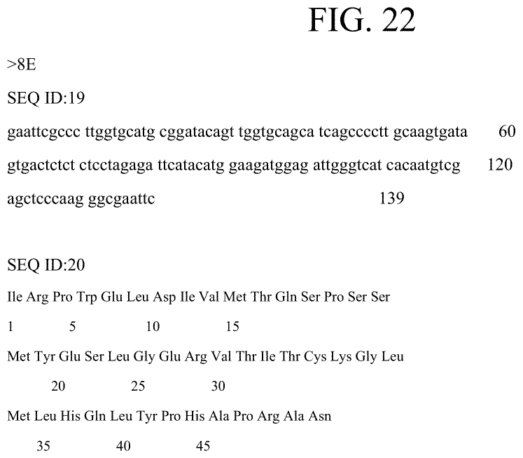

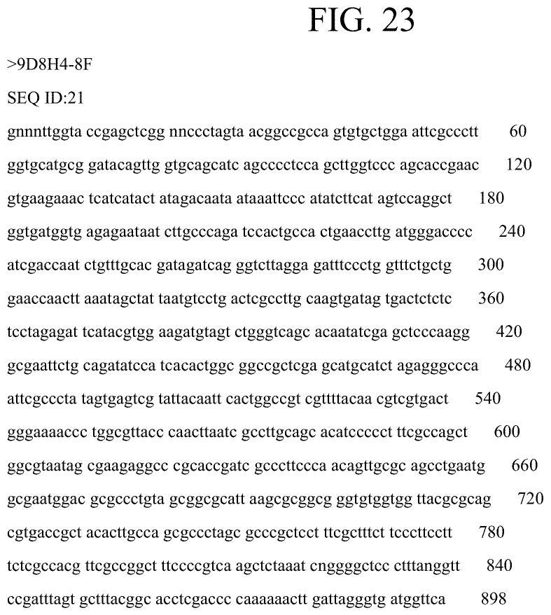

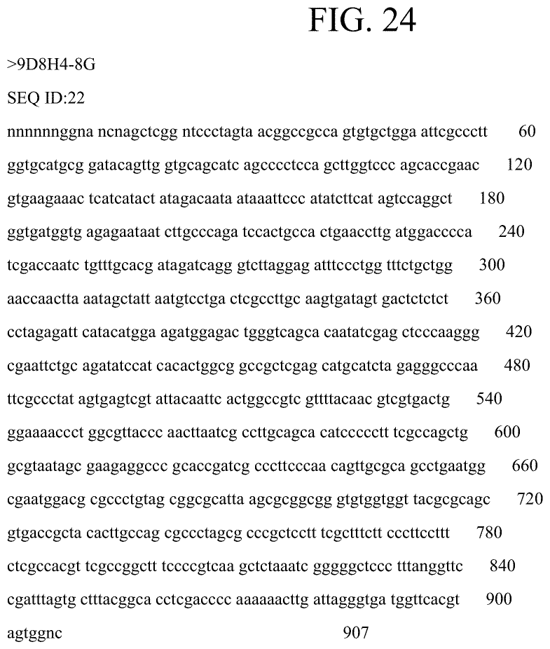

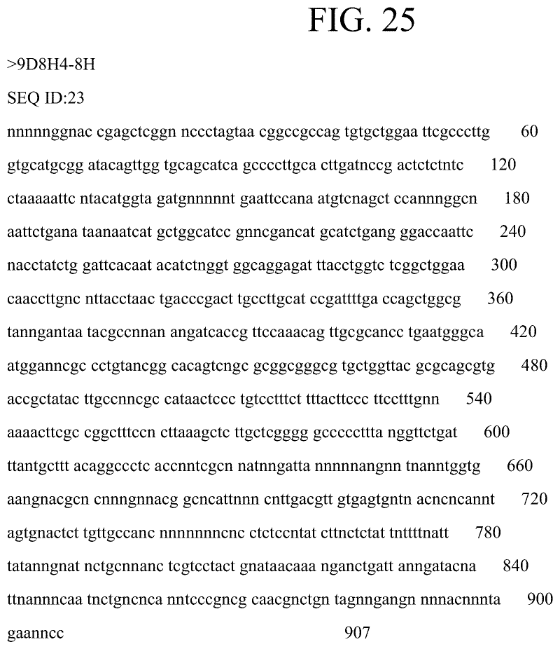

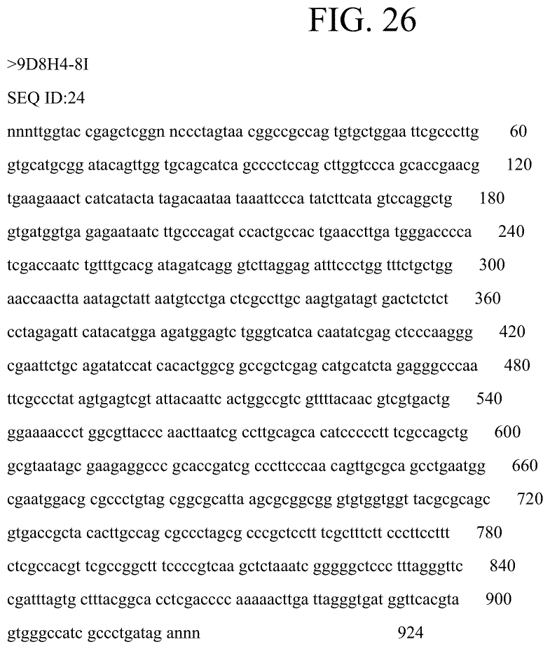

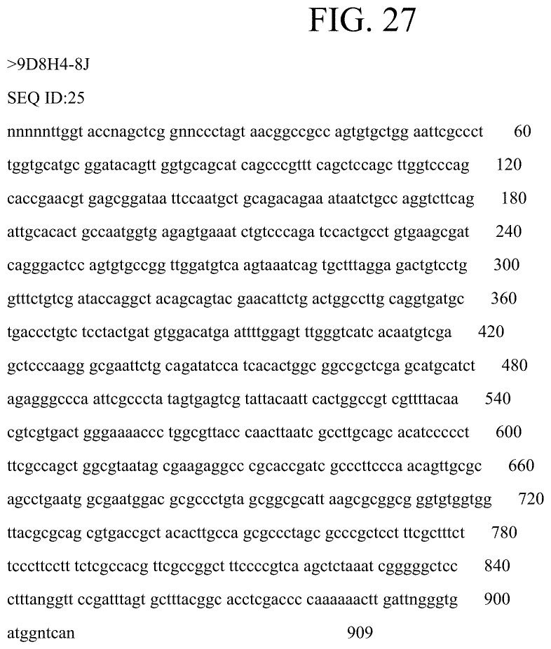

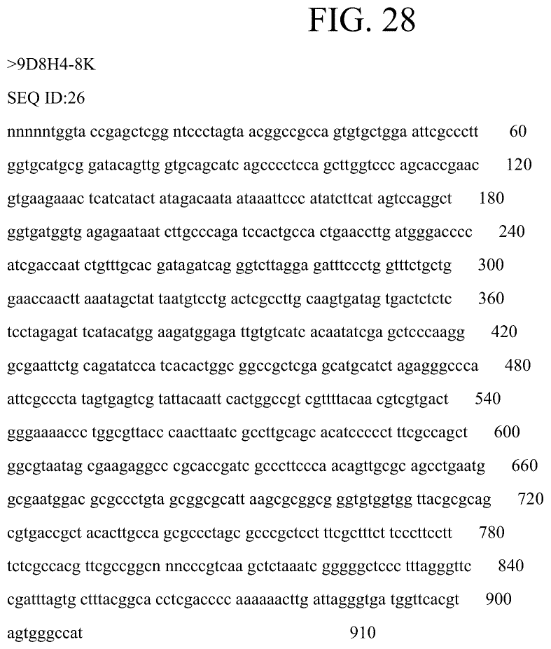

The light variable and heavy variable for amino acids and V region DNA sequences for mAb 16A1C8 are shown in FIG. 9. Specifically, SEQ ID NO:1, SEQ ID NO 2, SEQ ID NO:3 and SEQ ID NO4. Variations of mAb 9D8H4 are shown in FIGS. 10-28, which identify the amino acids and DNA sequences for. Specifically, SEQ ID NO:5, SEQ ID NO 6, SEQ ID NO:7, SEQ ID NO:8, SEQ ID NO 9, SEQ ID NO:10, SEQ ID NO:11, SEQ ID NO 12, SEQ ID NO:13, SEQ ID NO:14, SEQ ID NO 15, SEQ ID NO:16, SEQ ID NO:17, SEQ ID NO 18, SEQ ID NO:19, SEQ ID NO:20, SEQ ID NO 21, SEQ ID NO:22, SEQ ID NO:23, SEQ ID NO 24, SEQ ID NO:25 and SEQ ID NO:26.

### EXAMPLES

### Example 1

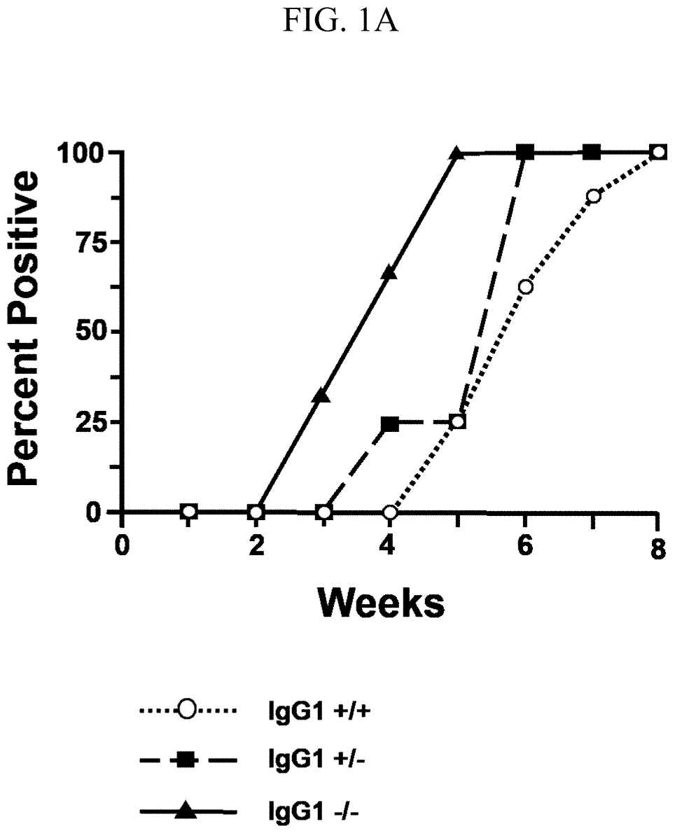

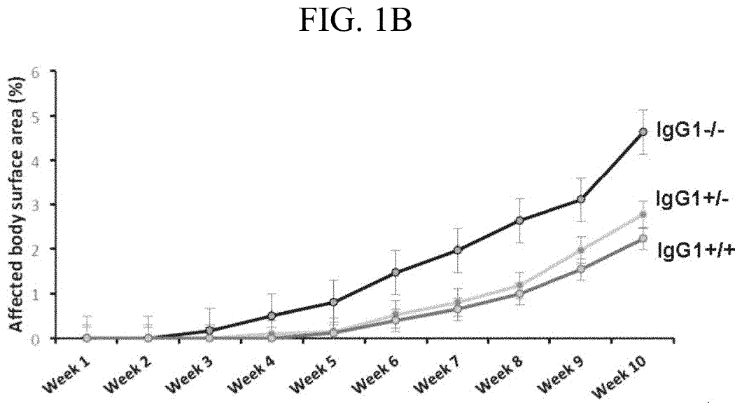

Eight week old male and female B6.s IgG1-deficient (IgG1−/−, 3/group), heterozygous (IgG1+/−, 4/group) and wild-type (IgG1+/+, 8/group) B6.s mice were inoculated into their footpads with 100 μg of murine Col7 vWFA2 peptide mixed with an equal volume of TiterMax adjuvant (total volume 60-100 μl) on day 0, and followed for 10 weeks for development of mEBA. FIGS. 1A and 1B show that IgG1 deficiency increases the risk of active mEBA. Percents of mice that developed skin blistering are shown in FIG. 1A; disease severity is shown in FIG. 1B. Error bars depict SEMs.

### Example 2

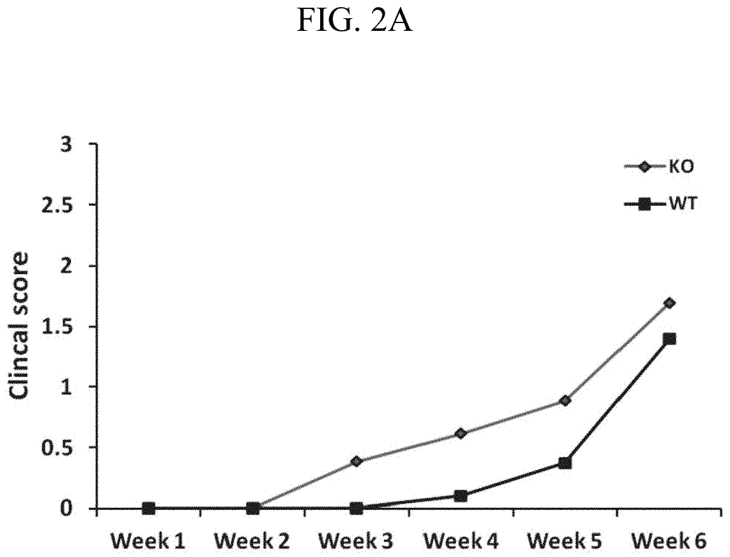

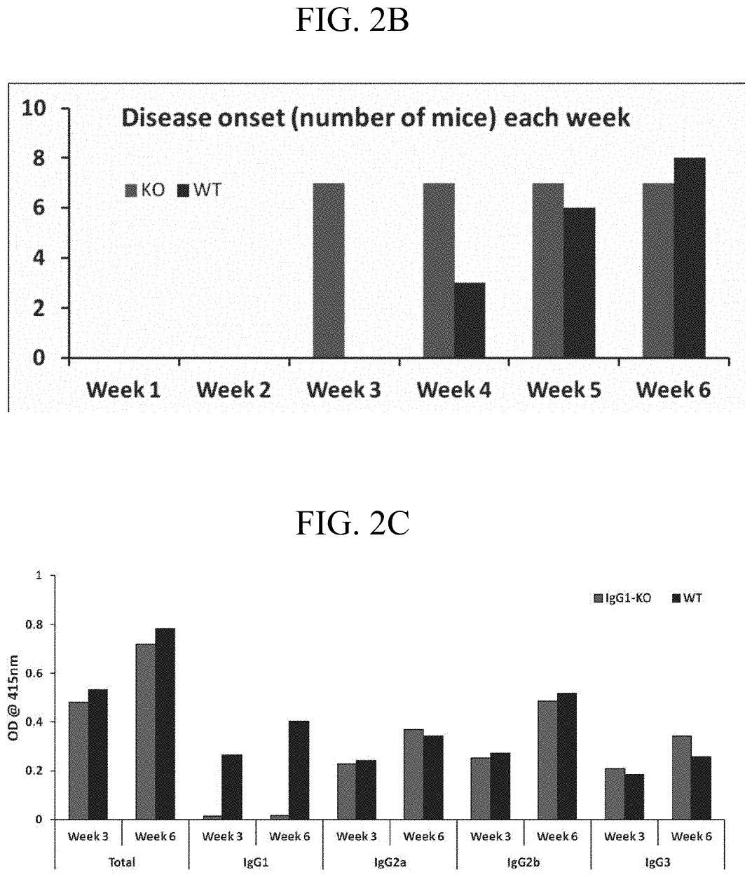

Eight week old male and female B6.s male and female IgG1-deficient (KO) and wild-type B6s (WT) mice (7-8/group) were inoculated in their footpads with 100 μg of murine Col7 vWFA2 peptide mixed with an equal volume of TiterMax adjuvant (total volume, 60-100 μl) on day 0, and followed for 6 weeks for development of mEBA. FIGS. 2A-2C show that IgG1 suppresses the development of active mEBA. Means of disease severity are shown in FIG. 2A; number of mice with detectable disease are shown in the FIG. 2B, and levels of serum total IgG, IgG1, IgG2a, IgG2b, and IgG3 anti-Col7 Ab are shown in FIG. 2C.

### Example 3

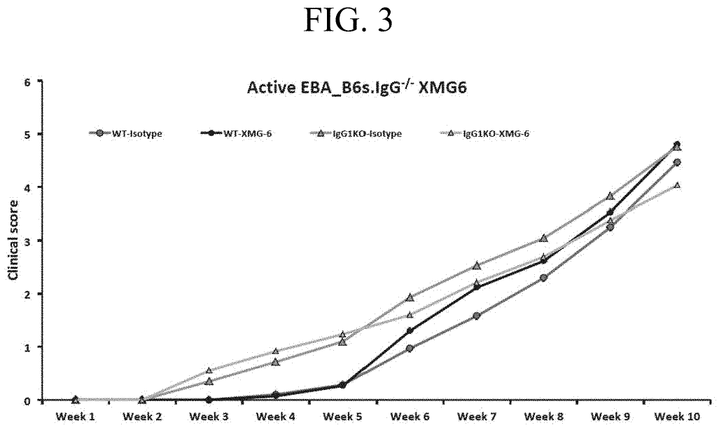

FIG. 3 shows that IFN-γ is not required for development of active mEBA. Eight week old male and female B6.s IgG1-deficient (B6s.IgG1−/−) and wild-type B6.s (WT) mice (6/group) were inoculated into their footpads with 100 μg of murine Col7 vWFA2 peptide mixed with an equal volume of TiterMax adjuvant (total volume 60-100 μl) on day 0, and with 100 μg intraperitoneally twice a week of XMG-6 rat IgG1 anti-mouse IFN-γ mAb or isotype control mAb and followed for 10 weeks for development of mEBA. Means of disease severity are shown in FIG. 3.

### Example 4

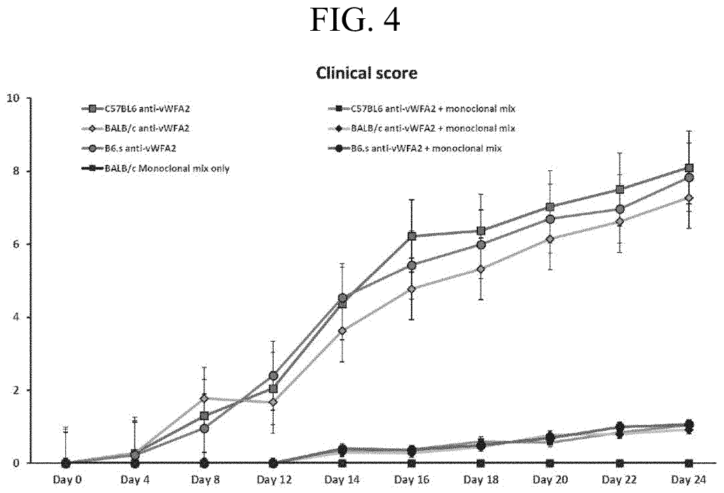

Eight week old male and female C57BL/6, B6.s, and BALB/c mice (4/group) were inoculated subcutaneously with 5 mg of RaCol7 Ab (anti-vWFA2) every other day, starting on day 0, and intraperitoneally every other day, starting on day −2, with 100 μg each of anti-Col7 mAb 16A1C8 and mAb 9D8H4 and followed for 24 days for development of mEBA. FIG. 4 shows suppression of induction of passive mEBA with a combination of two anti-mouse Col7 mAbs. Means and SEMs are shown.

### Example 5

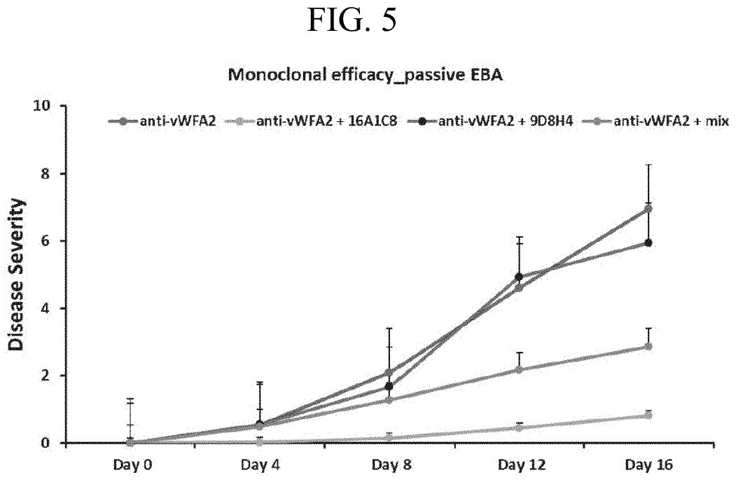

Eight to ten week old male and female C57BL/6 mice (6/group) were inoculated subcutaneously with 5 mg of RaCol7 Ab (anti-vWFA2) every other day, starting on day 0, and intraperitoneally every other day, starting on day 0, with 100 μg of anti-Col7 mAb 16A1C8, mAb 9D8H4, or with 50 μg of each mAb and followed for 16 days for development of mEBA. FIG. 5 is a graph showing that mAb 16A1C8, but not mAb 9D8H4, suppresses the passive model of mEBA. Means and SEMs are shown.

### Example 6

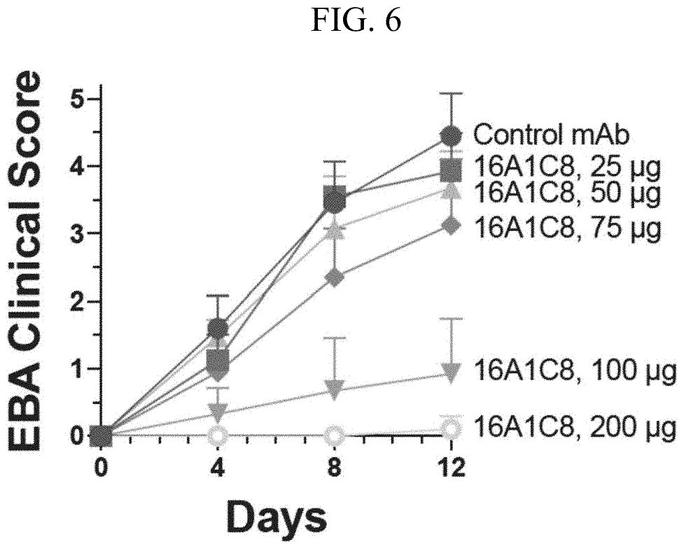

Eight to ten week old female C57BL6 mice (4/group) were inoculated subcutaneously with 5 mg of RaCol7 Ab (anti-vWFA2) every other day, starting on day 0, and intraperitoneally every other day, starting on day 0, with the doses shown of anti-Col7 mAb 16A1C8 and followed for the development of mEBA. FIG. 6 shows dose-related suppression of passive EBA by 16A1C8. Means and SEMs are shown.

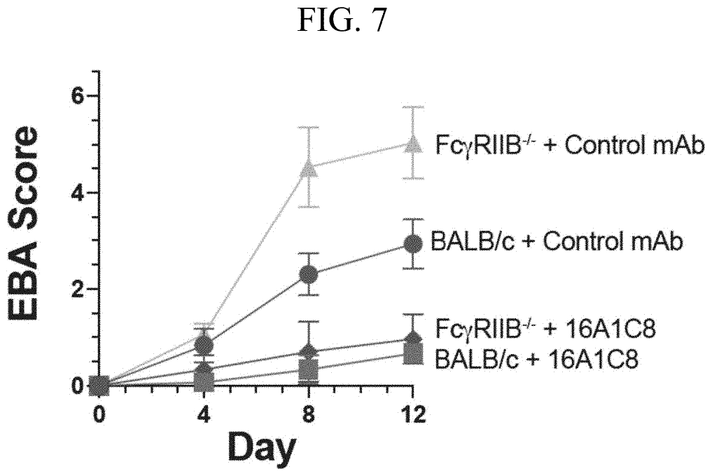

### Example 7

[71] Eight to ten week old male BALB/c wild-type mice (3/group) or male BALB/c-FcγRIIB−/− mice (3/group) were inoculated subcutaneously with 5 mg of rabbit anti-Col7 Ab (antivWFA2) IgG every other day, starting on day 0. Mice were also inoculated intraperitoneally every other day, starting on day 0, with 100 μg of anti-Col7 mAb 16A1C8 and followed for the development of mEBA. The dose of 16A1C8 was increased to 200 μg, starting on day 10. FIG. 7 shows that suppression of EBA Induction by mAb 16A1C8 is FcγRIIB-independent. Means and SEMs are shown.

### Example 8

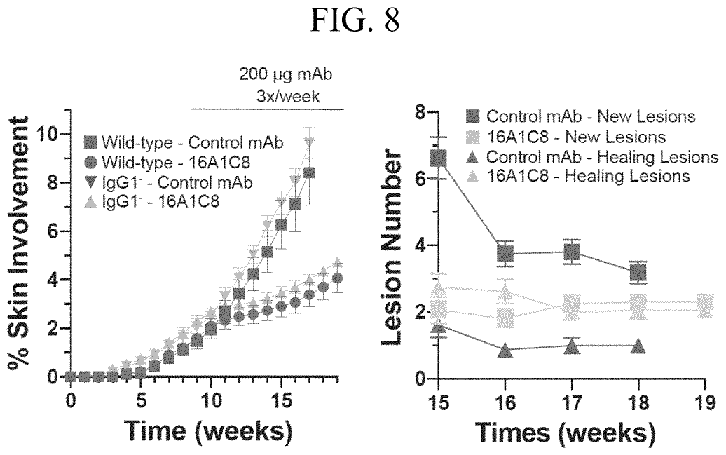

A test of an established active immunization model of mEBA is disclosed. Female wild-type or IgG1-deficient B6.s were actively immunized with a Col7 fragment emulsified in the nonionic block copolymer adjuvant TiterMax (Alexis Biochemicals) as in FIG. 1. Cutaneous lesions were counted, and the extent of skin disease was scored as the percentage of the skin surface that was involved and for whether lesions were progressing or healing. As with the passive model and our EBA studies, experiments tested the ability of 16A1C8 non-pathogenic anti-Col17 mAb (200 μg/week, starting 9 weeks after the initial Col to suppress established mEBA. As shown in FIG. 8, treatment with mAb 16A1C8 decreased disease progression and promoted lesion healing when administered to mice that had established mEBA. Regarding FIG. 8, note that lesions are larger in control mAb-treated mice than in 16A1C8-treated mice. Control mAb-treated mice were sacrificed at week 18.

### Example 9

Mucous membrane pemphigoid (MMP) is another human blistering skin disease that can be studied with mouse models. This disease, which predominantly affects the oral cavity, pharynx and conjunctiva, can be caused by autoantibodies to the 3 chain of laminin 332 (LAMα3). Injection of C57BL/6 mice with rabbit anti-LAMα3 causes skin erosions and crusting predominantly on the nose, conjunctiva, and ears. Rabbit anti-LAMα3 antiserum is generated by immunizing rabbits with mouse (m) LAMα3 peptides that contain amino acids 1656-1985 or 2756-3330 (generated as His fusion proteins). Mice are passively immunized every other day for 12 days by subcutaneous injection of up to 7.5 mg of the IgG fraction of the rabbit antiserum to induce the skin lesions. This experiment tests the ability of non-pathogenic anti-mLAMα3 mAbs to prevent rabbit anti-mLAMα3 induction of mMMP and determines whether the mAbs can suppress mMMP after disease induction with the rabbit antiserum.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of treating a subject suffering from a skin blistering disease, the method comprising:
a. identifying an epithelial cell adhesion molecule associated with the skin blistering disease (EpCAM);
b. identifying a non-pathogenic monoclonal antibody (mAb) that targets the EpCAM;
c. administering to the subject a therapeutically effective amount of the mAb.

2. The method of claim 1 wherein the skin disease is epidermolysis bullosa acquistita (EBA).

3. The method of claim 2 wherein the epithelial cell adhesion molecule is collagen type VII.

4. The method of claim 3 wherein the mAb is an IgG2c anti-collagen type VII mAb.

5. The method of claim 1 wherein the mAb is an antibody that binds to an epithelial cell adhesion molecule (EpCAM) comprising a heavy chain having the amino acid sequence SEQ ID NO:3, and a light chain having the amino acid sequence SEQ ID NO:1.

6. The method of claim 1 wherein the skin disease is bullous pemphigoid (BP).

7. The method of claim 6 wherein the epithelial cell adhesion molecule is collagen type XVII.

8. The method of claim 1, wherein the mAb further comprises a pharmaceutically acceptable carrier, excipient, flow agent, processing aid, diluent or a combination thereof.

9. A method of treating a subject with a skin blistering disease, the method comprising: administering to the subject a therapeutically effective amount of a pharmaceutical composition comprising non-pathogenic monoclonal antibodies (mAbs) and a pharmaceutically acceptable carrier excipient, flow agent, processing aid, diluent or a combination thereof to the subject.

10. The method of claim 9, wherein the mAb is an antibody that binds to an epithelial cell adhesion molecule (EpCAM) comprising a heavy chain having the amino acid sequence SEQ ID NO:3, and a light chain having the amino acid sequence SEQ ID NO:1.

11. A composition for the treatment of a skin blistering disease comprising a non-pathogenic monoclonal antibody (mAb), a pharmaceutically acceptable carrier, excipient, flow agent, processing aid, diluent or a combination thereof.

12. The composition of claim 11, wherein the mAb is an IgG2c anti-collagen type VII mAb.

13. The composition of claim 11, wherein the mAb is an antibody that binds to an epithelial cell adhesion molecule (EpCAM) comprising a heavy chain having the amino acid sequence SEQ ID NO:3, and a light chain having the amino acid sequence SEQ ID NO:1.

14. The composition of claim 11, wherein the composition is in a form suitable for oral, intravenous, intraaorterial, intramuscular, subcutaneous, parenteral, transmucosal, transdermal, ocular, or topical administration.

15. The composition of claim 11, wherein the composition is a topical application in the form of a cream, an ointment, a suspension, an emulsion, a gel or a combination thereof.
